(19) United States
(12) Patent Application Publication
(10) Pub. No.: US 2005/0075488 A1
(43) Pub. Date: Apr. 7, 2005

(54) Interleukin-1 beta antibodies

(76) Inventors: Stuart Willis Bright, Carmel, IN (US); Audrey Yunhua Jia, Union City, CA (US); Stuart Allen Kuhstoss, Indianapolis, IN (US); Joseph Vincent Manetta, Indianapolis, IN (US); Naoya Tsurushita, Palo Alto, CA (US); Maximiliano J. Vasquez, Palo Alto, CA (US)

Correspondence Address: ELI LILLY AND COMPANY;PATENT DIVISION, P.O. BOX 6288, INDIANAPOLIS, IN, 46206-6288, US

(21) Appl. No.: 10/484,280

(22) PCT Filed: Jul. 18, 2002
(86) PCT No.: PCT/US02/21281

Related U.S. Application Data
(60) Provisional application No. 60/307,973, filed on Jul. 26, 2001.
(60) Provisional application No. 60/312,278, filed on Aug. 14, 2001.

(51) Int. Cl. C07K 16/24

(52) U.S. Cl. 530/388.15; 530/388.23

(57) ABSTRACT

The present invention encompasses high affinity antibodies that neutralize IL-1β activity in vivo. These antibodies can be used to treat various diseases such as rheumatoid arthritis and osteoarthritis.

This application claims the benefit of U.S. Provisional Application Ser. No. 60/307973 filed Jul. 26, 2001, now abandoned and U.S. Provisional Application Ser. No. 60/312,278 filed Aug. 14, 2001, now abandoned.

Interleukin-1β (IL-1β) is a proinflammatory cytokine. IL-1β over-production has been implicated in the pathogeneis of a variety of diseases such as rheumatoid arthritis and osteoarthritis. IL-1β has been shown to increase cell migration into the inflamed synovium of joints by the up-regulation of adhesion molecules, the stimulation of the production of prostaglandins and metalloproteinase, the inhibition of collagen and proteoglycan synthesis, and the stimulation of osteoclastic bone resorption. Because of these properties, IL-1 is one of the primary mediators of bone and cartilage destruction in arthritis. Thus, agents that reduce the activity of IL-1β represent possible treatments for diseases such as arthritis.

There are three members of the IL-1 gene family: IL-1α, IL-1β, and IL-1 receptor antagonist (IL-1ra). IL-1α and IL-1β are agonists of the IL-1 receptor whereas the IL-1ra is a specific receptor antagonist and thus, an endogenous competitive inhibitor of IL-1. Administration of recombinant IL-1ra in clinical trials provided significant clinical improvements in patients with severe rheumatoid arthritis compared to placebo. Furthermore, administration of IL-1ra reduced the rate of progressive joint damage. However, the poor pharmacokinetic properties and the large dose that must be administered make recombinant IL-1ra a less than ideal therapeutic agent.

A high affinity neutralizing antibody to IL-1β would make a superior therapeutic agent. The typically long elimination half-lives of antibodies coupled with high affinity binding result in a therapeutic agent wherein much lower concentrations can be dosed much less frequently than recombinant IL-1ra. Although numerous IL-1β antibodies have been described, it has been exceedingly difficult to identify monoclonal antibodies having high affinity, high specificity, and potent neutralizing activity.

The present invention encompasses humanized IL-1β antibodies derived from a unique murine antibody to human IL-1β. These antibodies are high affinity antibodies that have potent IL-1β neutralizing activity and are highly specific for IL-1β.

This invention encompasses antibodies that specifically bind mature human IL-1β. The antibodies described and claimed herein bind the same epitope on mature human IL-1β as mouse monoclonal antibody Mu007 or humanized antibody Hu007. The invention includes antibodies that specifically bind mature human IL-1β with an affinity constant that is within ten-fold the affinity constant of mouse monoclonal antibody Mu007 for human IL-1β. The invention also includes antibodies, preferably humanized antibodies comprising at least one complementarity determining region having a sequence selected from the group consisting of SEQ ID NO:5, SEQ ID NO:6, SEQ ID NO:7, SEQ ID NO:8, SEQ ID NO:9, and SEQ ID NO:10. Most preferred are humanized antibodies comprising a light chain variable framework of human origin and three CDRs having sequences that correspond to SEQ ID NO:5, 6, and 7 and a heavy chain variable framework of human origin and three CDRs having sequences that correspond to SEQ ID NO:8, 9, and 10. The antibodies of the present invention include antibodies having framework regions that have at least 65% identity with the corresponding framework regions in mouse monoclonal antibody Mu007.

It is also preferred that the antibodies of the present invention have binding affinities within 10-fold that of mouse monoclonal antibody Mu007 or humanized antibody Hu007 and have potent neutralizing activity with IC50 values within 10-fold that of mouse monoclonal antibody Mu007 or humanized antibody Hu007.

The invention includes isolated nucleic acids comprising polynucleotides that encode the antibodies described and claimed herein. The invention also encompasses host cells transfected with these polynucleotides that express the antibodies described and claimed herein.

The invention encompasses methods of treating rheumatoid arthritis and osteoarthritis which comprise administering to a subject an effective amount of an antibody described and claimed herein as well as a method of inhibiting the destruction of cartilage that occurs in subjects that are prone to or have arthritis.

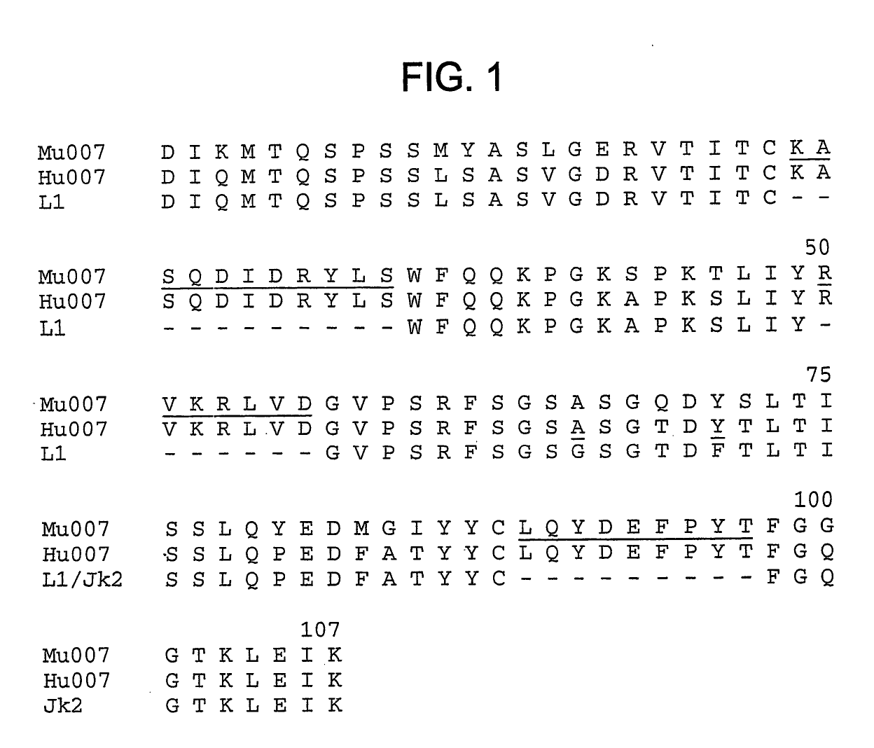

FIG. 1. Alignment of variable light chain amino acid sequences from Mu007, Hu007, and the germline L1 and JK2 segments. The CDR sequences based on the definition of Kabat are underlined in the Mu007 variable light chain sequence. The CDR sequences in the acceptor human variable light segment are omitted.

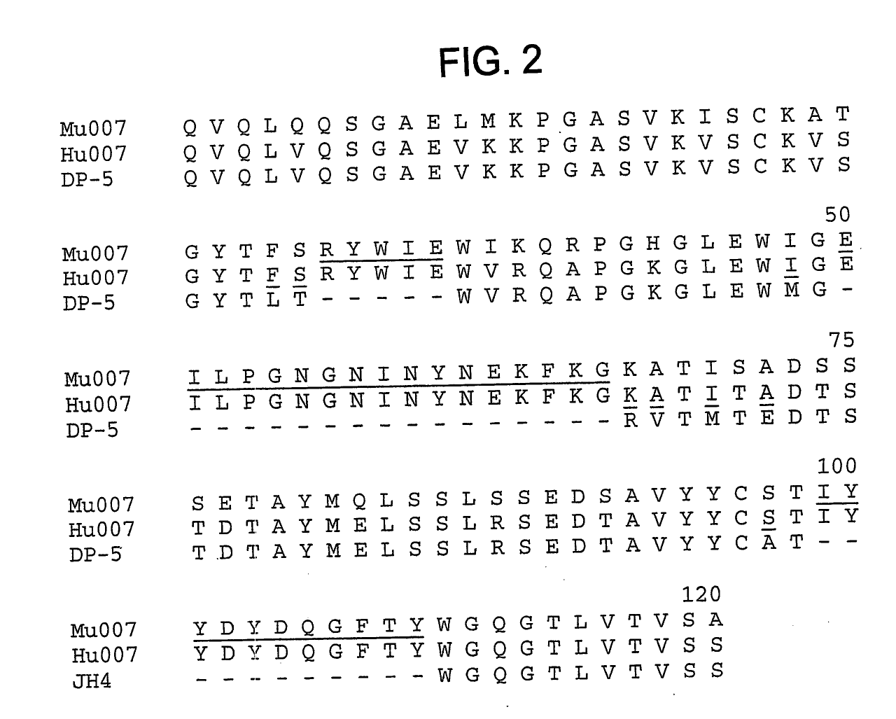

FIG. 2. Alignment of variable heavy chain amino acid sequences from Mu007, Hu007, and the germline DP5 and JH4 segments. The CDR sequences based on the definition of Kabat are underlined in the Mu007 variable heavy chain sequence. The CDR sequences in the acceptor human variable heavy segment are omitted.

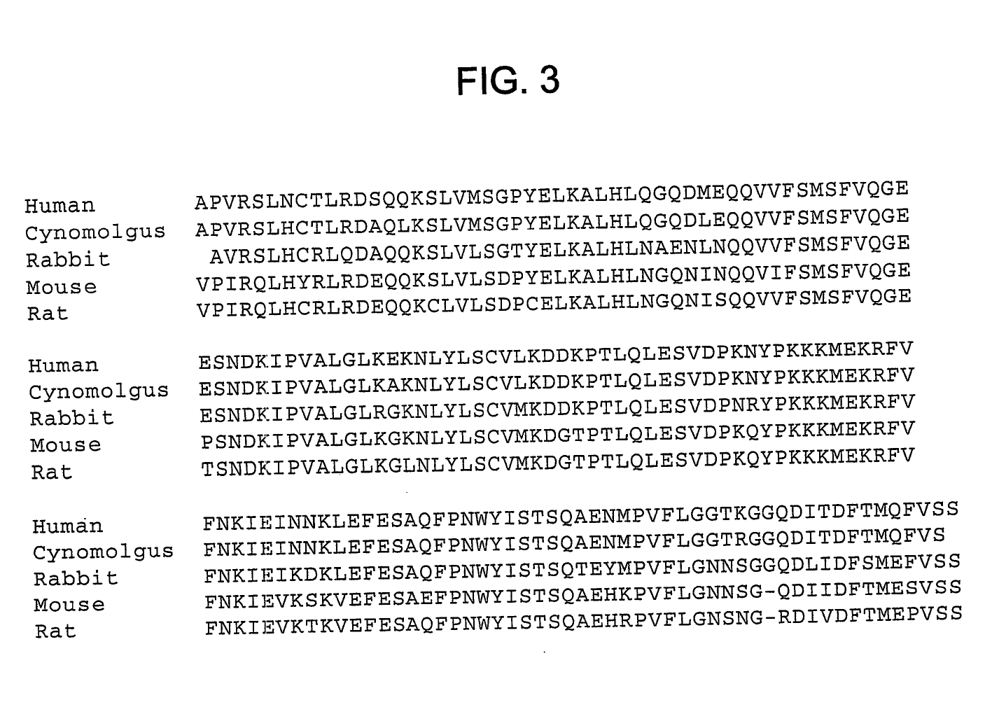

FIG. 3. Alignment of the mature IL-1β protein sequences from human, cynomolgous monkey, rabbit, rat, and mouse.

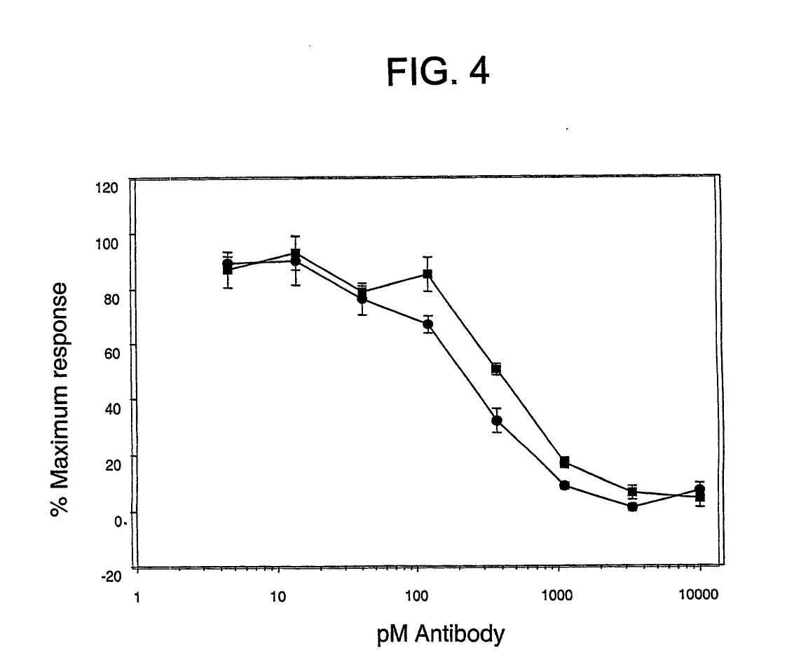

FIG. 4. Graph depicting the ability of Mu007 and Hu007 to inhibit the proliferation of an IL-1β-dependent cell line(●-Mu007; ▪-Hu007)

The present invention encompasses antibodies, preferably humanized antibodies, which bind the same epitope on human IL-1β as mouse monoclonal antibody Mu007. Preferably, these antibodies are comprised of the complementarity determining regions (CDRs) of the Mu007 antibody. The framework and other portions of these antibodies may originate from a human germ line. The humanized versions of the Mu007 antibody retain the high affinity, high specificity, and potent neutralizing activity observed for the Mu007 murine antibody.

As used herein, the word “treat” includes therapeutic treatment, where a condition to be treated is already known to be present and prophylaxis—i.e., prevention of, or amelioration of, the possible future onset of a condition.

A “subject” means a mammal, preferably a human having need of treatment. Subjects having need of treatment include mammals that are prone to arthritis, mammals that exhibit any cartilage destruction, and mammals that have signs and symptoms associated with rheumatoid arthritis or osteoarthritis.

An “isolated nucleic acid” is a nucleic acid molecule that is identified and separated from at least one contaminant nucleic acid molecule with which it is ordinarily associated in the natural source of the nucleic acid. Such an isolated nucleic acid molecule is other than in the form or setting in which it is found in nature. Isolated nucleic acid molecules therefore are distinguished from the nucleic acid molecule as it exists in natural cells. However, an isolated polypeptide(antibody)-encoding nucleic acid molecule includes polypeptide(antibody)-encoding nucleic acid molecules contained in cells that ordinarily express polypeptides where, for example, the nucleic acid molecule is in a chromosomal location different from that of natural cells.

“Antibody” means a complete antibody molecule, having full length heavy and light chains; a fragment thereof, such as a F_{ab}, F_{ab′}, or F_{(ab′)2} or Fv fragment; a single chain antibody fragment, e.g. a single chain Fv, a light chain or heavy chain monomer or dimer; multivalent monospecific antigen binding proteins comprising two, three, four, or more antibodies or fragments thereof bound to each other by a connecting structure; or an analogue of any of the above which binds the same epitope as mouse monoclonal antibody Mu007 or humanized antibody Hu007. In some contexts, herein, fragments will be mentioned specifically for emphasis; nevertheless, it will be understood that regardless of whether fragments are specified, the term “antibody” includes such fragments as well as single-chain forms. As long as the protein retains the ability to bind the same epitope on human IL-1β as Mu007 or Hu007, it is included within the term “antibody.” Preferably, but not necessarily, the antibodies useful in the invention are produced recombinantly. Antibodies may or may not be glycosylated, though glycosylated antibodies are preferred.

Antibodies that “specifically bind” mature human IL-1β include antibodies as defined above that bind the mature form of human IL-1β known in the art and represented in FIG. 3 and do not bind mature human IL-1α. An antibody that specifically binds mature human IL-1β may show some cross-reactivity with mature IL-1β from other species.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as kappa and lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 3 or more amino acids.

IgG antibodies are the most abundant immunoglobulin in serum. IgG also has the longest half-life in serum of any immunoglobulin. Unlike other immunoglobulins, IgG is efficiently recirculated following binding to FcRn. There are four IgG subclasses G1, G2, G3, and G4, each of which have different effector functions. G1, G2, and G3 can bind C1q and fix complement while G4 cannot. Even though G3 is able to bind C1q more efficiently than G1, G1 is more effective at mediating complement-directed cell lysis. G2 fixes complement very inefficiently. The C1q binding site in IgG is located at the carboxy terminal region of the CH2 domain.

All IgG subclasses are capable of binding to Fc receptors (CD16, CD32, CD64) with G1 and G3 being more effective than G2 and G4. The Fc receptor binding region of IgG is formed by residues located in both the hinge and the carboxy terminal regions of the CH2 domain.

IgA can exist both in a monomeric and dimeric form held together by a J-chain. IgA is the second most abundant Ig in serum, but it has a half-life of only 6 days. IgA has three effector functions. It binds to an IgA specific receptor on macrophages and eosinophils, which drives phagocytosis and degranulation, respectively. It can also fix complement via an unknown alternative pathway. IgM is expressed as either a pentamer or a hexamer, both of which are held together by a J-chain. IgM has a serum half-life of 5 days. It binds weakly to C1q via a binding site located in its CH3 domain. IgD has a half-life of 3 days in serum. It is unclear what effector functions are attributable to this Ig. IgE is a monomeric Ig and has a serum half-life of 2.5 days. IgE binds to two Fc receptors which drives degranulation and results in the release of proinflammatory agents.

Depending on the desired in vivo effect and the desired half-life, the antibodies of the present invention may contain any of the isotypes described above or may contain mutated regions wherein the complement and/or Fc receptor binding functions have been altered.

The variable regions of each light/heavy chain pair form the antibody binding site. Thus, an intact antibody has two binding sites. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with well known conventions [Kabat “sequences of Proteins of Immunological Interest” National Institutes of Health, Bethesda, Md., 1987 and 1991; Chothia, et al., J. Mol. Biol. 196:901-917 (1987); Chothia, et al., Nature 342:878-883 (1989)].

“Humanized antibody” means an antibody that is composed partially or fully of amino acid sequences derived from a human antibody germline or a rearranged sequence and made by altering the sequence of an antibody having non-human complementarity determining regions (CDR). The framework regions of the variable regions are substituted by corresponding human framework regions leaving the non-human CDR substantially intact. The framework region may be entirely human or may contain substitutions in regions that influence binding of the antibody to the target antigen. These regions may be substituted with the corresponding non-human amino acids. As discussed herein, antibody in the context of humanized antibody is not limited to a full-length antibody and can include fragments and single chain forms.

Humanized antibodies have several potential advantages over non-human and chimeric antibodies for use in human therapy. For example, the human immune system should not recognize the framework or constant region of the humanized antibody as foreign, and therefore the antibody response against such an injected antibody should be less than against a totally foreign non-human antibody or a partially foreign chimeric antibody. In addition, injected humanized antibodies generally have a longer half-life in the circulation than injected non-human antibodies. Furthermore, if effector function is desired, because the effector portion is human, it may interact better with the other parts of the human immune system.

Preferably, the antibodies of the present invention contain the CDRs from mouse antibody Mu007. The cDNA and amino acid sequence for the light chain variable region of the mouse MU007 antibody is as follows:

| | |
|---|---|
| GACATCAAGATGACCCAGTCTCCATCTTCCATGTATGCATCTCTAGGAGAGAGA | [SEQ ID NO:1] |
| D I K M T Q S P S S M Y A S L G E R | |
| GTCACTATCACTTGCAAGGCGAGTCAGGACATTGATAGGTATTTAAGTTGGTTCCAGCAG | |
| V T I T C K A S Q D I D R Y L S W F Q Q | |
| AAACCAGGGAAATCTCCTAAGACCCTGATCTATCGTGTAAAGAGATTGGTAGATGGGGTC | |
| K P G K S P K T L I Y R V K R L V D G V | |
| CCATCAAGGTTCAGTGGCAGCGCATCTGGGCAAGATTATTCTCTCACCATCAGCAGCCTG | |
| P S R F S G S A S G Q D Y S L T I S S L | |
| CAGTATGAAGATATGGGAATTTATTATTGTCTACAGTATGATGAGTTTCCGTACACGTTC | |
| Q Y E D M G I Y Y C L Q Y D E F P Y T F | |
| GGAGGGGGGACCAAGCTGGAAATAAAA | |
| G G G T K L E I K | |

The CDRs based on the definition of Kabat are underlined. The mature light chain begins with an aspartic acid residue. A signal sequence which can immediately precede SEQ ID NO:1 is as follows:

| | |
|---|---|
| ATGGACATGAGGACCCCTGCTCAGTTTCTTGGAATCTTTTTCTTCTGGTTTCCAGGTATC | [SEQ ID NO:19] |
| M D M R T P A Q F L G I F F F W F P G I | |
| AGATGT | |
| R C | |

The CDNA and amino acid sequence for the heavy chain variable region of the mouse Mu007 antibody is as follows:

| | |
|---|---|
| CAGGTTCAGCTGGTGCAGTCTGGAGCTGAGGTGAAGAAGCCTGGGGCCTCA | [SEQ ID NO:3] |
| Q V Q L V Q S G A E V K K P G A S | |
| GTGAAGGTGTCCTGCAAGGTGTCTGGCTACACATTCAGTAGGTATTGGATAGAGTGGGTT | |
| V K V S C K V S G Y T F S R Y W I E W V | |
| AGACAGGCACCTGGAAAAGGCCTTGAGTGGATTGGAGAGATTTTACCTGGAAATGGAAAT | |
| R Q A P G K G L E W I G E I L P G N G N | |
| ATTAACTACAATGAGAAGTTCAAGGGCAAGGCCACAATCACAGCAGATACATCCACAGAT | |
| I N Y N E K F K G K A T I T A D T S T D | |
| ACAGCCTACATGGAACTCAGCAGCCTGAGGTCTGAGGACACAGCCGTCTATTATTGTTCA | |
| T A Y M E L S S L R S E D T A V Y Y C S | |
| ACAATCTACTATGATTACGACCAGGGGTTTACTTACTGGGGCCAAGGGACTCTGGTCACT | |
| T I Y Y D Y D Q G F T Y W G Q G T L V T | |
| GTTTCTGCA | |
| V S A | |

The CDRs based on the definition of Kabat are underlined. The mature heavy chain begins with a glutamine residue. A signal sequence which can immediately precede SEQ ID NO:2 is as follows:

| | |
|---|---|
| ATGGAATGGACCTGGGTCTTTCTCTTCCTCCTGTCAGTAACTGCAGGTGTCCACTCC | [SEQ ID NO:21] |
| M E W T W V F L F L L S V T A G V H S | |

The preferred antibodies of the present invention have binding specificity, binding affinity, and potency similar to that observed for Mu007. The properties that define the antibodies of the present invention reside primarily in the variable regions of the antibody. Thus, the complete light chain and heavy chain variable regions of the Mu007 antibody can be used in the context of any constant region and the binding affinity and specificity as well as ability to neutralize mature human IL-1β will be generally unaffected. “Mu007” as used herein refers to the variable chain sequences represented as SEQ ID NO:1 and SEQ ID NO:3 in the context of any mouse constant region, preferably a kappa light chain and a gamma-1 heavy chain.

A preferred antibody of the present invention is a humanized antibody comprised of one or more CDRs with the following amino acid sequences:

| | |
|---|---|
| Light Chain CDR1: | |
| Lys Ala Ser Gln Asp Ile Asp Arg Tyr | [SEQ ID NO:5] |
| Leu Ser | |
| Light Chain CDR2: | |
| Arg Val Lys Arg Leu Val Asp | [SEQ ID NO:6] |
| Light Chain CDR3: | |
| Leu Gln Tyr Asp Glu Phe Tyr Thr | [SEQ ID NO:7] |
| Heavy Chain CDR1: | |
| Arg Tyr Trp Ile Glu | [SEQ ID NO:8] |
| Heavy Chain CDR2: | |
| Glu Ile Leu Pro Gly Asn Gly Asn Ile | [SEQ ID NO:9] |
| Asn Tyr Asn Glu Lys Phe Lys Gly | |
| Heavy Chain CDR3: | |
| Ile Tyr Tyr Asp Tyr Asp Gln Gly Phe | [SEQ ID NO:10] |
| Thr Tyr | |

In principle, a framework sequence from any human antibody may serve as the template for CDR grafting. However, straight chain replacement onto such a framework often leads to some loss of binding affinity to the antigen. The more homologous a human antibody is to the original murine antibody, the less likely the possibility that combining the murine CDRs with the human framework will introduce distortions in the CDRs that could reduce affinity. Therefore, it is preferable that the human variable framework that is chosen to replace the murine variable framework apart from the CDRs have at least a 65% sequence identity with the murine antibody variable region framework. It is more preferable that the human and murine variable regions apart from the CDRs have at least 70% sequence identify. It is even more preferable that the human and murine variable regions apart from the CDRs have at least 75% sequence identity. It is most preferable that the human and murine variable regions apart from the CDRs have at least 80% sequence identity. For example, a preferred human framework region for the variable light chain of the antibodies of the present invention as shown in FIG. 1 has approximately 80% sequence identity with the corresponding mouse sequence outside the CDRs. A preferred human framework region for the variable heavy chain of the antibodies of the present invention as shown in FIG. 2 has approximately 70% sequence identity with the corresponding mouse sequence outside the CDRs.

The heavy and light chain variable region framework residues can be derived from the same or different human antibody sequences. The human antibody sequences can be the sequences of naturally occurring human antibodies or can be consensus sequences of several human antibodies. Preferred human framework sequences for the heavy chain variable region of the humanized antibodies of the present invention include the VH segment DP-5 (Tomlinson, et al. (1992) J. Mol. Biol. 227:776-798) and the J segment JH4 (Ravetch, et al. (1981) Cell 27:583-591). The Vk segment L1 (Cox, et al. (1994) Eur. J. Immunol. 24:827-836) and the J segment Jk2 (Hieter, et al. (1982) J. Biol. Chem. 10:1516-1522) are preferred sequences to provide the framework for the humanized light chain variable region.

Certain amino acids from the human variable region framework residues were substituted with the corresponding murine amino acid to minimize effects on CDR conformation and/or binding to the IL-1β antigen.

Generally, when an amino acid falls under the following category, the framework amino acid of a human immunoglobulin to be used (acceptor immunoglobulin) is replaced by a framework amino acid from a CDR-providing non-human immunoglobulin (donor immunoglobulin):
(a) the amino acid in the human framework region of the acceptor immunoglobulin is unusual for human immunoglobulin at that position, whereas the corresponding amino acid in the donor immunoglobulin is typical for human immunoglobulin at that position;
(b) the position of the amino acid is immediately adjacent to one of the CDRs; or
(c) any side chain atom of a framework amino acid is within about 5-6 angstroms (center-to-center) of any atom of a CDR amino acid in a three dimensional immunoglobulin model [Queen, et al., Proc. Natl Acad. Sci. USA 86:10029-10033 (1989), and Co, et al., Proc. Natl. Acad. Sci. USA 88, 2869 (1991)]. When each of the amino acids in the human framework region of the acceptor immunoglobulin and a corresponding amino acid in the donor immunoglobulin is unusual for human immunoglobulin at that position, such an amino acid is replaced by an amino acid typical for human immunoglobulin at that position.

Analysis of the preferred framework regions for the humanized antibodies of the present invention suggested several amino acids that may have significant contact with the CDRs. These amino acids from mouse monoclonal antibody Mu007 were substituted for the original human framework amino acids.

FIGS. 1 and 2 provide an alignment of the variable light and heavy regions from the mouse sequence, a preferred humanized sequence, and a preferred human germline sequence. The single underlined amino acids in the humanized sequence were substituted with the corresponding mouse residues. For example, this was done at residues 29, 30, 48, 67, 68, 70, 72 and 97 of the heavy chain. For the light chain, the replacements were made at residues 66 and 71.

A preferred light chain variable region for the antibodies of the present invention comprises Formula I which is SEQ ID NO:27.

| | |
|---|---|
| 1 2 3 4 5 6 7 8 9 10 11 12 13 14 15 | Formula I [SEQ ID NO:27] |
| Asp Ile Xaa Met Thr Gln Xaa Pro Ser Ser Xaa Xaa Ala Ser Xaa | |
| 16 17 18 19 20 21 22 23 24 25 26 27 28 29 30 | |
| Gly Xaa Arg Val Thr Ile Thr Cys Lys Ala Ser Gln Asp Ile Asp | |
| 31 32 33 34 35 36 37 38 39 40 41 42 43 44 45 | |
| Arg Tyr Leu Ser Trp Phe Gln Gln Lys Pro Gly Lys Ala Pro Lys | |
| 46 47 48 49 50 51 52 53 54 55 56 57 58 59 60 | |
| Xaa Leu Ile Tyr Arg Val Lys Arg Leu Val Asp Gly Val Pro Ser | |
| 61 62 63 64 65 66 67 68 69 70 71 72 73 74 75 | |
| Arg Phe Ser Gly Ser Xaa Ser Gly Xaa Asp Tyr Thr Leu Thr Ile | |
| 76 77 78 79 80 81 82 83 84 85 86 87 88 89 90 | |
| Ser Ser Leu Gln Pro Glu Asp Phe Ala Thr Tyr Tyr Cys Leu Gln | |
| 91 92 93 94 95 96 97 98 99 100 101 102 103 104 105 | |
| Tyr Asp Glu Phe Pro Tyr Thr Phe Gly Gln Gly Thr Lys Leu Glu | |
| 106 107 | |
| Ile Lys | |
| Xaa at position 3 is Gln or Lys; | |
| Xaa at position 7 is Ser or Thr; | |
| Xaa at position 11 is Leu or Met; | |
| Xaa at position 12 is Ser, Tyr, or Thr; | |
| Xaa at position 15 is Val or Leu; | |
| Xaa at position 17 is Asp or Glu; | |
| Xaa at position 46 is Ser or Thr; | |
| Xaa at position 66 is Ala or Gly; and | |
| Xaa at position 69 is Thr or Gln; | |

A more preferred light chain variable region for the antibodies of the present invention comprises SEQ ID NO:11.

| | |
|---|---|
| Asp Ile Gln Met Thr Gln Ser Pro Ser Ser Leu Ser Ala Ser Val | [SEQ ID NO:11] |
| GAC ATC CAG ATG ACC CAG TCT CCA TCT TCC CTG TCT GCA TCT GTA | |
| Gly Asp Arg Val Thr Ile Thr Cys Lys Ala Ser Gln Asp Ile Asp | |
| GGA GAC AGA GTC ACT ATC ACT TGC AAG GCG AGT CAG GAC ATT GAT | |
| Arg Tyr Leu Ser Trp Phe Gln Gln Lys Pro Gly Lys Ala Pro Lys | |
| AGG TAT TTA AGT TGG TTC CAG CAG AAA CCA GGG AAA GCT CCT AAG | |
| Ser Leu Ile Tyr Arg Val Lys Arg Leu Val Asp Gly Val Pro Ser | |
| TCC CTG ATC TAT CGT GTA AAG AGA TTG GTA GAT GGG GTC CCA TCA | |
| Arg Phe Ser Gly Ser Ala Ser Gly Thr Asp Tyr Thr Leu Thr Ile | |
| AGG TTC AGT GGC AGC GCA TCT GGG ACA GAT TAT ACT CTC ACC ATC | |
| Ser Ser Leu Gln Pro Glu Asp Phe Ala Thr Tyr Tyr Cys Leu Gln | |
| AGC AGC CTG CAG CCT GAA GAT TTC GCA ACC TAT TAT TGT CTA CAG | |
| Tyr Asp Glu Phe Pro Tyr Thr Phe Gly Gln Gly Thr Lys Leu Glu | |
| TAT GAT GAG TTT CCG TAC ACG TTC GGA CAG GGG ACC AAG CTG GAA | |
| Ile Lys | |
| ATA AAA | |

A more preferred full-length light chain for the antibodies of the present invention comprises Formula II which is SEQ ID NO:13.

| | |
|---|---|
| Asp Ile Gln Met Thr Gln Ser Pro Ser Ser Leu Ser Ala Ser Val | [SEQ ID NO:13] |
| GAC ATC CAG ATG ACC CAG TCT CCA TCT TCC CTG TCT GCA TCT GTA | |
| Gly Asp Arg Val Thr Ile Thr Cys Lys Ala Ser Gln Asp Ile Asp | |
| GGA GAC AGA GTC ACT ATC ACT TGC AAG GCG AGT CAG GAC ATT GAT | |
| Arg Tyr Leu Ser Trp Phe Gln Gln Lys Pro Gly Lys Ala Pro Lys | |
| AGG TAT TTA AGT TGG TTC CAG CAG AAA CCA GGG AAA GCT CCT AAG | |
| Ser Leu Ile Tyr Arg Val Lys Arg Leu Val Asp Gly Val Pro Ser | |
| TCC CTG ATC TAT CGT GTA AAG AGA TTG GTA GAT GGG GTC CCA TCA | |
| Arg Phe Ser Gly Ser Ala Ser Gly Thr Asp Tyr Thr Leu Thr Ile | |
| AGG TTC AGT GGC AGC GCA TCT GGG ACA GAT TAT ACT CTC ACC ATC | |
| Ser Ser Leu Gln Pro Glu Asp Phe Ala Thr Tyr Tyr Cys Leu Gln | |
| AGC AGC CTG CAG CCT GAA GAT TTC GCA ACC TAT TAT TGT CTA CAG | |
| Tyr Asp Glu Phe Pro Tyr Thr Phe Gly Gln Gly Thr Lys Leu Glu | |
| TAT GAT GAG TTT CCG TAC ACG TTC GGA CAG GGG ACC AAG CTG GAA | |
| Ile Lys Arg Thr Val Ala Ala Pro Ser Val Phe Ile Phe Pro Pro | |
| ATA AAA CGA ACT GTG GCT GCA CCA TCT GTC TTC ATC TTC CCG CCA | |
| Ser Asp Glu Gln Leu Lys Ser Gly Thr Ala Ser Val Val Cys Leu | |
| TCT GAT GAG CAG TTG AAA TCT GGA ACT GCC TCT GTT GTG TGC CTG | |
| Leu Asn Asn Phe Tyr Pro Arg Glu Ala Lys Val Gln Trp Lys Val | |
| CTG AAT AAC TTC TAT CCC AGA GAG GCC AAA GTA CAG TGG AAG GTG | |
| Asp Asn Ala Leu Gln Ser Gly Asn Ser Gln Glu Ser Val Thr Glu | |
| GAT AAC GCC CTC CAA TCG GGT AAC TCC CAG GAG AGT GTC ACA GAG | |
| Gln Asp Ser Lys Asp Ser Thr Tyr Ser Leu Ser Ser Thr Leu Thr | |
| CAG GAC AGC AAG GAC AGC ACC TAC AGC CTC AGC AGC ACC CTG ACG | |
| Leu Ser Lys Ala Asp Tyr Glu Lys His Lys Val Tyr Ala Cys Glu | |
| CTG AGC AAA GCA GAC TAC GAG AAA CAC AAA GTC TAC GCC TGC GAA | |
| Val Thr His Gln Gly Leu Ser Ser Pro Val Thr Lys Ser Phe Asn | |
| GTC ACC CAT CAG GGC CTG AGC TCG CCC GTC ACA AAG AGC TTC AAC | |
| Arg Gly Glu Cys | |
| AGG GGA GAG TGT | |

A preferred signal sequence immediately preceding SEQ ID NO:11, 13, or 27 is as follows:

| | |
|---|---|
| Met Asp Met Arg Thr Pro Ala Gln Phe Leu Gly Ile Phe Phe Phe | [SEQ ID NO:23] |
| ATG GAC ATG AGG ACC CCT GCT CAG TTT CTT GGA ATC TTT TTC TTC | |
| Trp Phe Pro Gly Ile Arg Cys | |
| TGG TTT CCA GGT ATC AGA TGT | |

A preferred heavy chain variable region for the antibodies of the present invention comprises Formula II which is SEQ ID NO:28.

| | |
|---|---|
| 1 2 3 4 5 6 7 8 9 10 11 12 13 14 15 | Formula II [SEQ ID NO:28] |
| Xaa Val Gln Leu Val Gln Ser Gly Ala Glu Val Lys Lys Pro Gly | |
| 16 17 18 19 20 21 22 23 24 25 26 27 28 29 30 | |
| Ala Ser Val Lys Val Ser Cys Lys Xaa Ser Gly Tyr Thr Phe Xaa | |
| 31 32 33 34 35 36 37 38 39 40 41 42 43 44 45 | |
| Arg Tyr Trp Ile Glu Trp Xaa Arg Gln Ala Pro Gly Xaa Gly Leu | |
| 46 47 48 49 50 51 52 53 54 55 56 57 58 59 60 | |
| Glu Trp Xaa Gly Glu Ile Leu Pro Gly Asn Gly Asn Ile Asn Tyr | |
| 61 62 63 64 65 66 67 68 69 70 71 72 73 74 75 | |
| Asn Glu Lys Phe Lys Gly Xaa Xaa Thr Xaa Thr Ala Asp Xaa Ser | |
| 76 77 78 79 80 81 82 83 84 85 86 87 88 89 90 | |
| Xaa Xaa Thr Ala Tyr Met Glu Leu Ser Ser Leu Xaa Ser Glu Asp | |
| 91 92 93 94 95 96 97 98 99 100 101 102 103 104 105 | |
| Thr Ala Val Tyr Tyr Cys Ser Thr Ile Tyr Tyr Asp Tyr Asp Gln | |
| 106 107 108 109 110 111 112 113 114 115 116 117 118 119 120 | |
| Gly Phe Thr Tyr Trp Gly Gln Gly Thr Leu Val Thr Val Ser Ser | |
| Xaa at position 1 is Gln or Glu; | |
| Xaa at position 24 is Val, Ala, or Ser; | |
| Xaa at position 30 is Ser or Thr; | |
| Xaa at position 37 is Val or Ile; | |
| Xaa at position 43 is Lys, Gln, or His; | |
| Xaa at position 48 is Ile or Met; | |
| Xaa at position 67 is Lys or Arg; | |
| Xaa at position 68 is Ala or Val; | |
| Xaa at position 70 is Ile, Met, or Val; | |
| Xaa at position 74 is Thr or Ser; | |
| Xaa at position 76 is Thr or Ser; | |
| Xaa at position 77 is Asp, Glu, or Ser; and | |
| Xaa at position 87 is Arg or Ser | |

A more preferred heavy chain variable region for the antibodies of the present invention comprises SEQ ID NO:15.

| | |
|---|---|
| Gln Val Gln Leu Val Gln Ser Gly Ala Glu Val Lys Lys Pro Gly | [SEQ ID NO:15] |
| CAG GTT CAG CTG GTG CAG TCT GGA GCT GAG GTG AAG AAG CCT GGG | |
| Ala Ser Val Lys Val Ser Cys Lys Val Ser Gly Tyr Thr Phe Ser | |
| GCC TCA GTG AAG GTG TCC TGC AAG GTG TCT GGC TAC ACA TTC AGT | |
| Arg Tyr Trp Ile Glu Trp Val Arg Gln Ala Pro Gly Lys Gly Leu | |
| AGG TAT TGG ATA GAG TGG GTT AGA CAG GCA CCT GGA AAA GGC CTT | |
| Glu Trp Ile Gly Glu Ile Leu Pro Gly Asn Gly Asn Ile Asn Tyr | |
| GAG TGG ATT GGA GAG ATT TTA CCT GGA AAT GGA AAT ATT AAC TAG | |
| Asn Glu Lys Phe Lys Gly Lys Ala Thr Ile Thr Ala Asp Thr Ser | |
| AAT GAG AAG TTC AAG GGC AAG GCC ACA ATC ACA GCA GAT ACA TCC | |
| Thr Asp Thr Ala Tyr Met Glu Leu Ser Ser Leu Arg Ser Glu Asp | |
| ACA GAT ACA GCC TAC ATG GAA CTC AGC AGC CTG AGG TCT GAG GAC | |
| Thr Ala Val Tyr Tyr Cys Ser Thr Ile Tyr Tyr Asp Tyr Asp Gln | |
| ACA GCC GTC TAT TAT TGT TCA ACA ATC TAC TAT GAT TAC GAC CAG | |
| Gly Phe Thr Tyr Trp Gly Gln Gly Thr Leu Val Thr Val Ser Ser | |
| GGG TTT ACT TAC TGG GGC CAA GGG ACT CTG GTC ACT GTT TCT TCT | |

A more preferred full-length heavy chain region for the antibodies of the present invention comprises SEQ ID NO:17.

| | |
|---|---|
| Gln Val Gln Leu Val Gln Ser Gly Ala Glu Val Lys Lys Pro Gly | SEQ ID NO:17 |
| CAG GTT CAG CTG GTG CAG TCT GGA GCT GAG GTG AAG AAG CCT GGG | |
| Ala Ser Val Lys Val Ser Cys Lys Val Ser Gly Tyr Thr Phe Ser | |
| GCC TCA GTG AAG GTG TCC TGC AAG GTG TCT GGC TAC ACA TTC AGT | |
| Arg Tyr Trp Ile Glu Trp Val Arg Gln Ala Pro Gly Lys Gly Leu | |
| AGG TAT TGG ATA GAG TGG GTT AGA CAG GCA CCT GGA AAA GGC CTT | |
| Glu Trp Ile Gly Glu Ile Leu Pro Gly Asn Gly Asn Ile Asn Tyr | |
| GAG TGG ATT GGA GAG ATT TTA CCT GGA AAT GGA AAT ATT AAC TAC | |
| Asn Glu Lys Phe Lys Gly Lys Ala Thr Ile Thr Ala Asp Thr Ser | |
| AAT GAG AAG TTC AAG GGC AAG GCC ACA ATC ACA GCA GAT ACA TCC | |
| Thr Asp Thr Ala Tyr Met Glu Leu Ser Ser Leu Arg Ser Glu Asp | |
| ACA GAT ACA GCC TAC ATG GAA CTC AGC AGC CTG AGG TCT GAG GAC | |
| Thr Ala Val Tyr Tyr Cys Ser Thr Ile Tyr Tyr Asp Tyr Asp Gln | |
| ACA GCC GTC TAT TAT TGT TCA ACA ATC TAC TAT GAT TAC GAC CAG | |
| Gly Phe Thr Tyr Trp Gly Gln Gly Thr Leu Val Thr Val Ser Ser | |
| GGG TTT ACT TAC TGG GGC CAA GGG ACT CTG GTC ACT GTT TCT TCT | |
| Ala Ser Thr Lys Gly Pro Ser Val Phe Pro Leu Ala Pro Ser Ser | |
| GCC TCC ACC AAG GGC CCA TCG GTC TTC CCC CTG GCA CCC TCC TCC | |
| Lys Ser Thr Ser Gly Gly Thr Ala Ala Leu Gly Cys Leu Val Lys | |
| AAG AGC ACC TCT GGG GGC ACA GCG GCC CTG GGC TGC CTG GTC AAG | |
| Asp Tyr Phe Pro Glu Pro Val Thr Val Ser Trp Asn Ser Gly Ala | |
| GAC TAC TTC CCC GAA CCG GTG ACG GTG TCG TGG AAC TCA GGC GCC | |
| Leu Thr Ser Gly Val His Thr Phe Pro Ala Val Leu Gln Ser Ser | |
| CTG ACC AGC GGC GTG CAC ACC TTC CCG GCT GTC CTA CAG TCC TCA | |
| Gly Leu Tyr Ser Leu Ser Ser Val Val Thr Val Pro Ser Ser Ser | |
| GGA CTC TAC TCC CTC AGC AGC GTG GTG ACC GTG CCC TCC AGC AGC | |
| Leu Gly Thr Gln Thr Tyr Ile Cys Asn Val Asn His Lys Pro Ser | |
| TTG GGC ACC CAG ACC TAC ATC TGC AAC GTG AAT CAC AAG CCC AGC | |
| Asn Thr Lys Val Asp Lys Lys Val Glu Pro Lys Ser Cys Asp Lys | |
| AAC ACC AAG GTG GAC AAG AAA GTT GAG CCC AAA TCT TGT GAC AAA | |
| Thr His Thr Cys Pro Pro Cys Pro Ala Pro Glu Leu Leu Gly Gly | |
| ACT CAC ACA TGC CCA CCG TGC CCA GCA CCT GAA CTC CTG GGG GGA | |
| Pro Ser Val Phe Leu Phe Pro Pro Lys Pro Lys Asp Thr Leu Met | |
| CCG TCA GTC TTC CTC TTC CCC CCA AAA CCC AAG GAC ACC CTC ATG | |
| Ile Ser Arg Thr Pro Glu Val Thr Cys Val Val Val Asp Val Ser | |
| ATC TCC CGG ACC CCT GAG GTC ACA TGC GTG GTG GTG GAC GTG AGC | |
| His Glu Asp Pro Glu Val Lys Phe Asn Trp Tyr Val Asp Gly Val | |
| CAC GAA GAC CCT GAG GTC AAG TTC AAC TGG TAC GTG GAC GGC GTG | |
| Glu Val His Asn Ala Lys Thr Lys Pro Arg Glu Glu Gln Tyr Asn | |
| GAG GTG CAT AAT GCC AAG ACA AAG CCG CGG GAG GAG CAG TAC AAC | |
| Ser Thr Tyr Arg Val Val Ser Val Leu Thr Val Leu His Gln Asp | |
| AGC ACG TAC CGT GTG GTC AGC GTC CTC ACC GTC CTG CAC CAG GAC | |
| Trp Leu Asn Gly Lys Glu Tyr Lys Cys Lys Val Ser Asn Lys Ala | |
| TGG CTG AAT GGC AAG GAG TAC AAG TGC AAG GTC TCC AAC AAA GCC | |
| Leu Pro Ala Pro Ile Glu Lys Thr Ile Ser Lys Ala Lys Gly Gln | |
| CTC CCA GCC CCC ATC GAG AAA ACC ATC TCC AAA GCC AAA GGG CAG | |
| Pro Arg Glu Pro Gln Val Tyr Thr Leu Pro Pro Ser Arg Asp Glu | |
| CCC CGA GAA CCA CAG GTG TAC ACC CTG CCC CCA TCC CGG GAT GAG | |
| Leu Thr Lys Asn Gln Val Ser Leu Thr Cys Leu Val Lys Gly Phe | |
| CTG ACC AAG AAC CAG GTC AGC CTG ACC TGC CTG GTC AAA GGC TTC | |
| Tyr Pro Ser Asp Ile Ala Val Glu Trp Glu Ser Asn Gly Gln Pro | |
| TAT CCC AGC GAC ATC GCC GTG GAG TGG GAG AGC AAT GGG CAG CCG | |
| Glu Asn Asn Tyr Lys Thr Thr Pro Pro Val Leu Asp Ser Asp Gly | |
| GAG AAC AAC TAC AAG ACC ACG CCT CCC GTG CTG GAC TCC GAC GGC | |
| Ser Phe Phe Leu Tyr Ser Lys Leu Thr Val Asp Lys Ser Arg Trp | |
| TCC TTC TTC CTC TAC AGC AAG CTC ACC GTG GAC AAG AGC AGG TGG | |
| Gln Gln Gly Asn Val Phe Ser Cys Ser Val Met His Glu Ala Leu | |
| CAG CAG GGG AAC GTC TTC TCA TGC TCC GTG ATG CAT GAG GCT CTG | |
| His Asn His Tyr Thr Gln Lys Ser Leu Ser Leu Ser Pro Gly Lys | |
| CAC AAC CAC TAC ACG CAG AAG AGC CTC TCC CTG TCT CCG GGT AAA | |

A preferred signal sequence immediately preceding SEQ ID NO:15, 17, or 28 is the following:

| | |
|---|---|
| Met Glu Trp Thr Trp Val Phe Leu Phe Leu Leu Ser Val | [SEQ ID NO:25] |
| ATG GAA TGG ACC TGG GTC TTT CTC TTC CTC CTG TCA GTA | |
| Thr Ala Gly Val His Ser | |
| ACT GCA GGT GTC CAC TCC | |

Antibody “Hu007” as referred to herein is a humanized version of mouse monoclonal antibody Mu007 having a light chain sequence corresponding to SEQ ID NO:13 and a heavy chain sequence corresponding to SEQ ID NO:17.

The primary impetus for humanizing antibodies from another species is to reduce the possibility that the antibody causes an immune response when injected into a human patient as a therapeutic. The more human sequences that are employed in a humanized antibody, the lower the risk of immunogenicity. Changes can be made to the sequences described herein as preferable heavy and light chain regions without significantly affecting the biological properties of the antibody. This is especially true for the antibody constant regions and parts of the variable region which do not influence the ability of the CDRs to bind to IL-1β.

Furthermore, as discussed herein other human framework variable regions and variants thereof may be used in the present invention. However, regardless of the framework chosen, if reducing the risk of immunogenicity is a focus, the number of changes relative to the human framework chosen should be minimized.

The present invention encompasses antibodies or other proteins that make use of one or more of the CDRs of antibody Mu007. The CDRs encompassed by the present invention are the hypervariable regions of the Mu007 antibody which provide the majority of contact residues for the binding of the antibody to a specific IL-1β epitope. Thus, the CDRs described herein can be used to make full-length antibodies as well as functional fragments and analogs or other proteins which when attached to the CDRs maintain the CDRs in an active structural conformation such that the binding affinity of the protein employing the CDRs for mature IL-1β increases compared to the binding affinity of Mu007, is the same as the binding affinity of Mu007, or does not decrease by more than 10-fold compared to the binding affinity of the Mu007 antibody or alternatively, is not less than 10-fold less compared to the binding affinity of the Mu007 antibody. Preferably the binding affinity does not decrease by more than 5-fold compared to the binding affinity of the Mu007 antibody. Most preferably the binding affinity is within 3-fold the binding affinity of the Mu007 antibody.

The binding affinity of the Mu007 antibody was determined using surface plasmon resonance (BIAcore™). In these experiments antibody was immobilized at low density on a BIAcore™ chip and ligand was flowed past. Build up of mass at the surface of the chip was measured. This analytical method allows the determination in real time of both on and off rates for binding. The Mu007 antibody has an affinity of approximately 6.0 picomolar (See Example 9). A preferred humanized antibody of the present invention, Hu007 had an affinity of approximately 10 to 20 picomolar. (See example 9). Hu007 comprises heavy and light chains that correspond to SEQ ID NO:17 and SEQ ID NO:13, respectively.

It is also preferred that the antibodies or other proteins of the present invention which employ the CDRs of the Mu007 antibody bind specifically to IL-1β and not other IL-1 family members or structurally related proteins within the same species. For example, neither the Mu007 nor Hu007 bind to human IL-1α. (See example 9).

It is also preferred that the antibodies or other proteins of the present invention which employ the CDRs of the Mu007 antibody neutralize the biological activity of IL-1β. Two different assays were employed to test the ability of Mu007 and Hu007 to neutralize IL-1β activity. A murine cell line which requires low levels of IL-1β for proliferation was used in the first assay. Human IL-1β was present at a constant level in the medium and a dilution series of each antibody was added. Inhibition of proliferation provided a measurement of the efficacy of the antibody's ability to block IL-1β activation of the IL-1 receptor. Proliferation measurements for different concentrations of antibody resulted in an average IC50 value of 220 picomolar for Mu007 and 480 picomolar for Hu007 (See example 10). It is preferred that the antibodies or other proteins of the present invention have an IC50 potency which is better than, the same as, or within 10-fold that of Mu007 or alternatively, not less than 10-fold lower than that of MU007. Preferably the IC50 potency is within 5-fold that of Mu007. Most preferably the IC50 potency is within 3-fold that of Mu007. “IC50” as referred to herein is the measure of potency of an antibody to inhibit the activity of human IL-1β. IC50 is the concentration of antibody that results in 50% IL-1β inhibition in a single dose experiment. The IC50 can be measured by any assay that detects inhibition of human IL-1β activity. However, the IC50 values obtained may vary depending on the assay used. There may even be some variability between experiments using the same assay. For example, the condition of the IL-1β dependent cells discussed herein, has an effect on the IC50 values obtained. Thus, the critical value for the purposes of the present invention is a value relative to that obtained using Mu007 or Hu007 in a single experiment.

Neither Mu007 nor Hu007 cross-react with mouse IL-1β making it difficult to use a mouse model to test neutralizing activity in vivo. However, one consequence of the proinflammatory activity of IL-1β is the induction of IL-6, another proinflammatory cytokine that mediates some of the non-local effects of IL-1β. Human IL-1β is able to bind and stimulate the mouse IL-1β receptor, leading to an elevation of mouse IL-6. Thus, an antibody with neutralizing activity would block the induction of IL-6 in a mouse given a dose of human IL-1β. Both Mu007 and Hu007 demonstrated potent neutralization of human IL-1β in the murine model of inflammatory stimulation. The humanized antibody was approximately ⅓ as efficacious as the Mu007 antibody (See example 11).

The invention also encompasses antibodies wherein the Mu007 CDRs have been grafted into a human framework region or a human framework variant such as in Hu007 and then modified or mutated to enhance binding affinity or other biological properties such as the ability of the antibody to neutralize IL-1β activity at specific concentrations which can be expressed as an IC50 value.

It is preferred that the antibodies of the present invention bind the same epitope on human IL-1β as the Mu007 and Hu007 antibodies. In addition, the invention encompasses antibodies that bind epitopes that overlap with or include the epitope bound by the Mu007 and Hu007 antibodies provided those antibodies have the ability to neutralize human IL-1β in vivo.

The present invention encompasses the discovery of a specific region of the 153 amino acid mature form of human IL-1β wherein the binding of an antibody to that region completely neutralizes activity of the protein. Furthermore, antibodies directed to this specific region of mature IL-1β are specific in that they do not cross react with other IL-1 family members or related proteins. While the invention encompasses all antibodies that bind this epitope and neutralize IL-1β activity, it is preferred that the antibodies employ at least one of the CDRs present in Mu007. Antibodies that neutralize IL-1β activity prevent the mature IL-1β protein from binding to its receptor and/or initiating a signal transduction pathway.

The IL-1β epitope bound by Mu007 and/or Hu007 can be determined by providing a family of fragments containing different amino acid segments from the mature IL-1β protein. Each fragment typically comprises at least 4, 6, 8, 10, 20, 50, or 100 contiguous amino acids. The family of polypeptide fragments cover much or all of the amino acid sequence of mature IL-1β. Members of the family are tested individually for binding to the Mu007 or Hu007 antibodies. The smallest fragment that can specifically bind to the antibody being tested contains the amino acid sequence of the epitope recognized by the antibody. An additional method to map epitopes involves testing the ability of an antibody to bind IL-1β in which random mutations have been introduced. This method may be preferred if the epitope's three-dimensional structure is critical for binding. Because neither Mu007 nor Hu007 bind mouse or rat IL1β, the epitope recognized by Mu007 or Hu007 maps to an area or areas of the IL-1β protein which is not completely conserved between the mouse, rat, and human sequences. Further, Mu007 and Hu007 bind and neutralize Cynomolgus and rabbit IL-1β. Therefore, the epitope recognized by Mu007 and Hu007 must be largely conserved among human, Cynomolgus, and rabbit IL-1β. FIG. 3 depicts an alignment of the mouse, rat, rabbit, Cynomolgus, and human mature IL-1β protein sequences. Thus, to map the epitope recognized by Mu007 and Hu007, mutations can be targeted to sites that show sequence conservation among human, Cynomolgus, and rabbit IL-1β but which differ in mouse and rat IL-1β. Positions which fulfill these conditions include valine 3, serine 5, glycine 22, glutamate 51, aspartate76, lysine 77, isoleucine 106, leucine 110, methionine 130, glycine 140, and glutamate 150 (FIG. 3, numbering according to the human or Cynomolgus sequence. Mutating aspartate 76 to glycine and lysine 77 to threonine has no effect on binding to Cynomolgus IL-1β. Therefore this region is not important for binding of Mu007 and Hu007 to IL-1β.

IL-1β can also be captured by immobilized antibody and the complex treated with proteases such as trypsin to cleave portions of the molecule that are not protected by the antibody. After digestion, unbound peptides are washed away. The remaining bound peptides are eluted from the antibody and subjected to mass spectrometric analysis to determine their identity. Alternatively, IL-1β can be digested with proteases, and peptides can be captured by antibody. Unbound peptides are washed away. The remaining bound peptide is eluted from the antibody and subjected to mass spectrometric analysis to determine its identity.

An additional epitope mapping experiment involves the use of NMR spectroscopy. IL-1β protein is expressed in a host cell such as E. coli grown in medium enriched for Nitrogen¹⁵, carbon , and deuterium. Labeled IL-1β is purified and analyzed by NMR. NMR peaks are assigned to different amino acids. The analysis is then repeated in the presence of Fabs derived from IL-1 β antibody. A change in specific NMR peaks is indicative of a different environment of the amino acids contributing to the peaks. This could be due to binding of antibody to the specific amino acids, to physical proximity of the antibody to the amino acids, or to a conformational shift induced by antibody binding which leads to an altered environment for the specific amino acids. Often it is the case, especially when a conformational epitope is mapped, that more than one method is applied to confirm a predicted antibody binding site.

The epitopic fragment which binds Mu007 and Hu007 can be used as an immunogen to obtain additional crossreacting antibodies with high affinity binding and potent neutralizing activity which can be used directly or humanized for use as a therapeutic agent.

The present invention also is directed to recombinant DNA encoding antibodies which, when expressed, specifically bind to the same epitope that Mu007 and Hu007 bind to and have potent in vivo neutralizing activity. Preferably, the DNA encodes antibodies that, when expressed, comprise one or more of the heavy and light chain Mu007 CDRs [SEQ ID NO:5,6,7,8,9, and 10]. Exemplary DNA sequences which, on expression, code for the polypeptide chains comprising the heavy and light chain CDRs of the Mu007 and Hu007 antibodies are represented as SEQ ID NO:1, 3, 11, 13, 15, and 17. Due to the degeneracy of the genetic code, other DNA sequences can be readily substituted for the exemplified sequences.

DNA encoding the antibodies of the present invention will typically further include an expression control polynucleotide sequence operably linked to the antibody coding sequences, including naturally-associated or heterologous promoter regions. Preferably, the expression control sequences will be eukaryotic promoter systems in vectors capable of transforming or transfecting eukaryotic host cells, but control sequences for prokaryotic hosts may also be used. Once the vector has been incorporated into the appropriate host cell line, the host cell is propagated under conditions suitable for expressing the nucleotide sequences, and, as desired, the collection and purification of the light chains, heavy chains, light/heavy chain dimers or intact antibodies, binding fragments or other immunoglobulin forms.

The nucleic acid sequences of the present invention capable of ultimately expressing the desired antibodies can be formed from a variety of different polynucleotides (genomic or cDNA, RNA, synthetic oligonucleotides, etc.) and components (e.g., V, J, D, and C regions), using any of a variety of well known techniques. Joining appropriate genomic and synthetic sequences is a common method of production, but cDNA sequences may also be utilized.

Human constant region DNA sequences can be isolated in accordance with well known procedures from a variety of human cells, but preferably from immortalized B-cells. Suitable source cells for the polynucleotide sequences and host cells for immunoglobulin expression and secretion can be obtained from a number of sources well-known in the art.

As described herein, in addition to the antibodies specifically described herein, other “substantially homologous” modified antibodies can be readily designed and manufactured utilizing various recombinant DNA techniques well known to those skilled in the art. For example, the framework regions can vary from the native sequences at the primary structure level by several amino acid substitutions, terminal and intermediate additions and deletions, and the like. Moreover, a variety of different human framework regions may be used singly or in combination as a basis for the humanized immunoglobulins of the present invention. In general, modifications of the genes may be readily accomplished by a variety of well-known techniques, such as site-directed mutagenesis.

Alternatively, polypeptide fragments comprising only a portion of the primary antibody structure may be produced, which fragments possess one or more immunoglobulin activities (e.g., complement fixation activity). These polypeptide fragments may be produced by proteolytic cleavage of intact antibodies by methods well known in the art, or by inserting stop codons at the desired locations in vectors using site-directed mutagenesis, such as after CH1 to produce Fab fragments or after the hinge region to produce F(ab′)₂ fragments. Single chain antibodies may be produced by joining VL and VH with a DNA linker.

As stated previously, the polynucleotides will be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors will contain selection markers, e.g., tetracycline, neomycin, and dihydrofolate reductase, to permit detection of those cells transformed with the desired DNA sequences.

E. coli is a prokaryotic host useful particularly for cloning the polynucleotides of the present invention. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any of a number of well-known promoters may be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

In addition to microorganisms, Plant cells may also be used for expression. Optimal methods of plant transformation vary depending on the type of plant. For example, see WO0053794 which is herein incorporated by reference.

Mammalian tissue cell culture may also be used to express and produce the polypeptides of the present invention. Eukaryotic cells are actually preferred, because a number of suitable host cell lines capable of secreting intact immunoglobulins have been developed in the art, and include the CHO cell lines, various COS cell lines, Syrian Hamster Ovary cell lines, HeLa cells, myeloma cell lines, transformed B-cells, human embryonic kidney cell lines, or hybridomas. Preferred cell lines are CHO and myeloma cell lines such as SP2/0 and NS0.

Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, cytomegalovirus and the like. Preferred polyadenylation sites include sequences derived from Sv40 and bovine growth hormone.

The vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts.

Once expressed, the antibodies can be purified according to standard procedures, including ammonium sulfate precipitation, ion exchange, affinity (e.g. Protein A), reverse phase, hydrophobic interaction column chromatography, gel electrophoresis, and the like. Substantially pure immunoglobulins having at least about 90 to 95% purity are preferred, and 98 to 99% or more purity most preferred, for pharmaceutical uses. Once purified, partially or to homogeneity as desired, the polypeptides may then be used therapeutically or prophylactically, as directed herein.

This invention also relates to a method of treating humans experiencing an IL-1β mediated inflammatory disorder which comprises administering an effective dose of an IL-1β antibody to a patient in need thereof. The antibodies of the present invention bind to and prevent IL-1β from binding an IL-1β receptor and initiating a signal. Various IL-1β-mediated disorders include rheumatoid arthritis (RA), osteoarthritis (OA), Allergy, septic or endotoxic shock, septicemia, stroke, asthma, graft versus host disease, Crohn's disease, and other inflammatory bowel diseases. Preferably, the IL-1β antibodies encompassed by the present invention are used to treat RA and/or OA.

Patients with RA suffer from chronic swelling and inflammation of the joints and ongoing destruction of cartilage and bone. IL-1β and TNF-α are the most critical cytokines in the pathogenesis of RA. However, while both IL-1β and TNF-α mediate inflammation, IL-1β is the primary mediator of bone and cartilage destruction. Activated monocytes and fibroblasts in the synovial tissue produce IL-1β which in turn stimulates the production of additional pro-inflammatory cytokines, prostaglandins, and matrix metalloproteases. The synovial lining becomes hypertrophied, invading and eroding bone and cartilage.

Disease-modifying antirheumatic drugs (DMARDS) such as hydroxychloroquine, oral or injectable gold, methotrexate, azathioprine, penicillamine, and sulfasalazine have been used with modest success in the treatment of RA. Their activity in modifying the course of RA is believed to be due to suppression or modification of inflammatory mediators such as IL-1β. Methotrexate, for example, at doses of 7.5 to 10 mg per week caused a reduction in IL-1β plasma concentrations in RA patients. Similar results have been seen with corticosteroids. Thus, the IL-1β antibodies of the present invention may be used alone or in combinations with DMARDS which may act to reduce IL-1β protein levels in plasma.

An effective amount of the IL-1β antibodies of the present invention is that amount which provides clinical efficacy without intolerable side effects or toxicity. Clinical efficacy for RA patients can be assessed using the American College of Rheumatology Definition of Improvement (ACR20). A patient is considered a responder if the patient shows a 20% improvement in the tender joint count, swollen joint count, and 3 of 5 other components which include patient pain assessment, patient global assessment, physician global assessment, Health Assessment Questionnaire, and serum C-reactive protein. Prevention of structural damage can be assessed by the van der Heijde modification of the Sharp Scoring system for radiographs (erosion count, joint space narrowing).

The IL-1β antibodies of the present invention can also be used to treat patients suffering from osteoarthritis (OA). OA is the most common disease of human joints and is characterized by articular cartilage loss and osteophyte formation. Clinical features include joint pain, stiffness, enlargement, instability, limitation of motion, and functional impairment. OA has been classified as idiopathic (primary) and secondary forms. Criteria for classification of OA of the knee and hip have been developed by the American College of Rheumatology on the basis of clinical, radiographic, and laboratory parameters.

An effective amount of the IL-1β antibodies of the present invention is the amount which shows clinical efficacy in OA patients as measured by the improvement in pain and function as well as the prevention of structural damage. Improvements in pain and function can be assessed using the pain and physical function subscales of the WOMAC OA Index. The index probes clinically important patient-relevant symptoms in the areas of pain, stiffness, and physical function. Prevention of structural damage can be assessed by measuring joint space width on radiographs of the knee or hip.

The antibodies of the present invention are administered using standard administration techniques, preferably peripherally (i.e. not by administration into the central nervous system) by intravenous, intraperitoneal, subcutaneous, pulmonary, transdermal, intramuscular, intranasal, buccal, sublingual, or suppository administration.

The pharmaceutical compositions for administration are designed to be appropriate for the selected mode of administration, and pharmaceutically acceptable excipients such as, buffers, surfactants, preservatives, solubilizing agents, isotonicity agents, stabilizing agents and the like are used as appropriate. Remington's Pharmaceutical Sciences, Mack Publishing Co., Easton Pa., latest edition, incorporated herein by reference, provides a compendium of formulation techniques as are generally known to practitioners.

The concentration of the IL-1β antibody in formulations may be from as low as about 0.1% to as much as 15 or 20% by weight and will be selected primarily based on fluid volumes, viscosities, stability, and so forth, in accordance with the particular mode of administration selected. Preferred concentrations of the IL-1β antibody will generally be in the range of 1 to about 100 mg/mL. Preferably, 10 to about 50 mg/mL.

The formulation may include a buffer. Preferably the buffer is a citrate buffer or a phosphate buffer or a combination thereof. Generally, the pH of the formulation is, between about 4 and about 8. Preferably, the pH is between about 5 and about 7.5. The pH of the formulation can be selected to balance antibody stability (chemical and physical) and comfort to the patient when administered. The formulation may also include a salt such as NaCl. In addition, the formulation may include a detergent to prevent aggregation and aid in maintaining stability. For example, Tween-80 and Tween-20 were shown to be compatible with the Hu007 antibody.

The formulation may be sterile filtered after making. the formulation, or otherwise made microbiologically acceptable. A preservative such as m-cresol or phenol, or a mixture thereof may be added to prevent microbial growth and contamination.

A typical composition for intravenous infusion could have a volume as much as 250 mL of fluid, such as sterile Ringer's solution, and 1-100 mg per mL, or more in antibody concentration. Therapeutic agents of the invention can be frozen or lyophilized for storage and reconstituted in a suitable sterile carrier prior to use. Lyophilization and reconstitution can lead to varying degrees of antibody activity loss (e.g. with conventional immunoglobulins, IgM antibodies tend to have greater activity loss than IgG antibodies). Dosages may have to be adjusted to compensate.

Although the foregoing methods appear the most convenient and most appropriate for administration of proteins such as humanized antibodies, by suitable adaptation, other techniques for administration, such as transdermal administration and oral administration may be employed provided proper formulation is designed. In addition, it may be desirable to employ controlled release formulations using biodegradable films and matrices, or osmotic mini-pumps, or delivery systems based on dextran beads, alginate, or collagen. In summary, formulations are available for administering the antibodies of the invention and may be chosen from a variety of options.

Typical dosage levels can be optimized using standard clinical techniques and will be dependent on the mode of administration and the condition of the patient. Generally, doses will be in the range of 10 μg/kg/month to 10 mg/kg/month.

The invention is illustrated by the following examples which are not intended to be limiting in any way.

### EXAMPLE 1

Mu007 Variable Regions

The Mu007 light and heavy chain variable region cDNAs were cloned from a hybridoma cell line. Several light and heavy chain clones were sequenced from two independent PCR reactions. The functional light chain variable sequence was typical of a functional mouse kappa chain variable region and was found to belong to subgroup V based on the definition of Kabat (Johnson, G. and Wu, T. T. (2000) Nucleic Acids Res. 28: 214-218). For the heavy chain, a unique sequence homologous to a typical mouse heavy chain variable region was identified. Mu007 variable heavy chain was classified to subgroup II(A) based on the definition of Kabat (Johnson and Wu, 2000). The cDNA sequences coding light and heavy chain variable regions are represented as SEQ ID NO:1 and 3, respectively.

### EXAMPLE 2

Hu007 Variable Regions

The human variable region framework used as an acceptor for Mu007·CDRs was constructed and amplified using eight overlapping synthetic oligonucleotides ranging in length from approximately 65 to 80 bases (He, et al. (1998) J. Immunol. 160: 1029-1035). The oligonucleotides were annealed pairwise and extended with the Klenow fragment of DNA polymerase I, yielding four double-stranded fragments. The resulting fragments were denatured, annealed pairwise, and extended with Klenow, yielding two fragments. These fragments were denatured, annealed pairwise, and extended once again, yielding a full-length gene. The PCR-amplified fragments were gel-purified and cloned into pCR4Blunt vector. After sequence confirmation, the variable light and variable heavy genes were digested with M1uI and XbaI, gel-purified, and subcloned respectively into vectors for expression of light and heavy chains to make pVk-Hu007 and pVg1-Hu007.

### EXAMPLE 3

Expression of Hu007

Mouse myeloma cell line Sp2/0-Ag14 (hereinafter, Sp2/0) was obtained from the ATCC and maintained in DME medium containing 10% FBS (Cat # SH30071.03, Hyclone, Logan, Ut.) in a 37° C. incubator.

Stable transfection into mouse myeloma cell line Sp2/0 was accomplished by electroporation using a Gene Pulser apparatus (BioRad, Hercules, Calif.) at 360 V and 25 μF according to the manufacturer's instructions. Before transfection, pVk-Hu007 and pVg1-Hu007 plasmid DNAs were linearized using FspI. Approximately 107 Sp2/0 cells were transfected with 30 μg of pVk-Hu007 and 60 μg of pVg1-Hu007. The transfected cells were suspended in DME medium containing 10% FBS and plated into several 96-well plates. After 48 hr, cells were selected for gpt expression using selection media (DME medium containing 10% FBS, HT media supplement, 0.3 mg/ml xanthine and 1 μg/ml mycophenolic acid). Approximately 10 days after the initiation of selection, culture supernatants were assayed for antibody production by ELISA (See Example 7). High-yielding clones were expanded in DME medium containing 10% FBS and further analyzed for antibody expression. Selected clones were then adapted to growth in serum free medium (Hybridoma SFM, Cat. # 12045-076, Life Technologies, Rockville, Md). This was accomplished by splitting the cells gradually in Hybridoma SFM, usually by a 25 to 50% split each time, until the serum level was below 0.1%. Thereafter, the transfectant was maintained in Hybridoma SFM. The cell density was maintained between 2×10⁵/ml and 10⁶/ml.

CHO-DG44 cells were transfected with 50 μg of pVk-Hu007 and 50 μg of pVg1-Hu007 (genomic transfection) or 50 μg of an expression vector containing cDNA corresponding to the Hu007 light chain and 50 μg of a vector containing cDNA corresponding to the Hu007 heavy chain. Approximately 10⁷ cells were electroporated at 350 volts/50 μF and 380 volts/50 μF for the genomic transfection and 350 volts/71 μF and 380 volts/71 μF for the cDNA transfection. Cells were incubated at room temperature and then diluted with 20 ml Growth Medium (ExCell 302 medium+4 mM L-Glutamine+1×hypoxanthine/thymidine reagent+100 μg/mL dextran sulfate) and allowed to recover for 72 hours in a 37° C./5% CO₂ incubator. Cells were selected with medium containing 50 nM methotrexate for the genomic transfectants and 20 nM methotrexate and 200 μg/mL G418 for the cDNA transfectants.

### EXAMPLE 4

Purification of Hu007

A high expressing Sp2/0 clone was expanded to 1,500 ml in Hybridoma SFM in roller bottles (500 ml per roller bottle). Hu007 IgG1 monoclonal antibody was purified from spent culture supernatant with a protein-A Sepharose column. Spent culture supernatant was harvested when cell viability reached 10% or below and loaded onto a protein-A Sepharose column. The column was washed with PBS before the antibody was eluted with 0.1 M glycine-HCl (pH 2.8), 0.1 M NaCl. After neutralization with 1 M Tris HCl (pH 8), the eluted protein was dialyzed against 3 changes of 2 liters PBS and filtered through a 0.2 Am filter prior to storage at 4° C. absorbance at 280 nm (1 mg/ml =1.4 A₂₈₀).

### EXAMPLE 5

Expression and Purification of Mu007

Hybridoma cells producing Mu007 were first grown in RPMI-1640 medium containing 10% FBS (HyClone), 10 mM HEPES, 2 mM glutamine, 0.1 mM non-essential amino acids, 1 mM sodium pyruvate, 25 μg/ml gentamicin, and then expanded in serum-free media (Hybridoma SFM, Cat # 12045-076, Life Technologies, Rockville, Md.) containing 2% low Ig FBS (Cat # 30151.03, HyClone) to a 1 liter volume in roller bottles. Mu007 was purified from the culture supernatant by affinity chromatography using a protein-G Sepharose column. Biotinylated Mu007 was prepared using EZ-Link Sulfo-NHS-LC-LC-Biotin (Cat # 21338ZZ, Pierce, Rockford, Il.).

### EXAMPLE 6

SDS-PAGE Analysis of Isolated Mu007 and Hu007

SDS-PAGE in Tris-glycine buffer was performed according to standard procedures on a 4-20% gradient gel (Cat # EC6025, Novex, San Diego, Calif.).

SDS-PAGE analysis of Mu007 and Hu007 under non-reducing conditions indicated that both antibodies have a molecular weight of about 150-160 kD. Analysis under reducing conditions indicated that both antibodies were comprised of a heavy chain with a molecular weight of about 50 kD and a light chain with a molecular weight of about 25 kD. The purity of Hu007 appeared to be more than 95%.

### EXAMPLE 7

Quantification of Antibody Expression by ELISA

Wells of 96-well ELISA plates (Nunc-Immuno plate, Cat # 439454, NalgeNunc, Naperville, Il.) were coated with 100 μl of 1 μg/ml goat anti-human IgG, Fc□ fragment specific, polyclonal antibodies (Cat # 109-005-098, Jackson ImmunoResearch, West Grove, Pa.) in 0.2 M sodium carbonate-bicarbonate buffer (pH 9.4) overnight at 4° C. After washing with Washing Buffer (PBS containing 0.1% Tween 20), wells were blocked with 400 μl of Superblock Blocking Buffer (Cat # 37535, Pierce) for 30 min and then washed with Washing Buffer. Samples containing Hu007 were appropriately diluted in ELISA Buffer (PBS containing 1% BSA and 0.1% Tween 20) and applied to ELISA plates (100 μl per well). As a standard, humanized anti-CD33 IgG1 monoclonal antibody HuM195 (Co et al. (1992) J. Immunol. 148: 1149-1154) was used. ELISA plates were incubated for 2 hr at 37° C. and the wells were washed with Wash Buffer. Then, 100 μl of 1/1,000-diluted HRP-conjugated goat anti-human kappa polyclonal antibodies (Cat # 1050-05, Southern Biotechnology, Birmingham, Al.) in ELISA Buffer was applied to each well. After incubating for 1 hr at 37° C. and washing with Wash Buffer, 100 μl of ABTS substrate (Cat #s 507602 and 506502, Kirkegaard and Perry Laboratories, Gaithersburg, Md.) was added to each well. Color development was stopped by adding 100 μl of 2% oxalic acid per well. Absorbance was read at 415 nm using an OPTImax microplate reader (Molecular Devices, Menlo Park, Calif.).

### EXAMPLE 8

ELISA Competition

Wells of 96-well ELISA plates (Nunc-Immuno plate, Cat # 439454, NalgeNunc) were coated with 100 μμl of 0.5 μg/ml of human IL-1β in 0.2M sodium carbonate-bicarbonate buffer (pH 9.4) overnight at 4° C., washed with Wash Buffer, blocked with Superblock blocking buffer for 30 min at 37° C., and washed again with Wash Buffer. A mixture of biotinylated Mu007 (0.16 μg/ml final concentration) and competitor antibody (Mu007 or Hu007; starting at 100 μg/ml final concentration and serial 3-fold dilutions) in ELISA Buffer were added in triplicate in a final volume of 100 μl per well. As a no-competitor control, 100 μl of 0.16 μg/ml biotinylated Mu007 was used. As a background control, 100 μl of ELISA Buffer was used. ELISA plates were incubated at 37° C. for 2 hr. After washing the wells with Washing Buffer, 100 μl of 1 μg/ml HRP-conjugated streptavidin (Jackson ImmunoResearch) was added to each well. ELISA plates were incubated at room temperature for 30 min and washed with Washing Buffer. For color development, 100 μl/well of ABTS substrate was added. Color development was stopped by adding 100 μl/well of 2% oxalic acid. Absorbance was read at 415 nm.

Both Mu007 and Hu007 competed with biotinylated Mu007 in a concentration-dependent manner. A concentration-dependant competition indicates that the competing antibodies bind the same epitope on mature IL-1β. The IC₅₀ values of Mu007 and Hu007 in three independent experiments, obtained using the computer software Prism (GraphPad Software Inc., San Diego, Calif.) are shown in Table 1. The relative binding of Hu007 was on average 1.3 fold less than that of Mu007.

TABLE 1
Summary of ELISA competition experiments
IC₅₀ (μg/ml)

| | | | | | Std. |
|---|---|---|---|---|---|
| Competitor | Exp. A | Exp. B | Exp. C | Average | Dev. |
| Mu007 | 0.40 | 0.40 | 0.39 | 0.40 | 0.0069 |
| Hu007 | 0.39 | 0.35 | 0.32 | 0.35 | 0.035 |
| Difference | 0.98 | 0.88 | 0.82 | 0.89 | |

### EXAMPLE 9

Binding Affinity and Specificity

Affinities and specificities of both Hu007 and Mu007 were determined using BIAcore measurements. BIAcore™ is an automated biosensor system that measures molecular interactions. (Karlsson, et al. (1991) J. Immunol. Methods 145: 229-240). In these experiments antibody was immobilized at low density on a BIAcoreTm chip. Ethyl-dimethylaminopropyl-carbodiimide (EDC) was used to couple reactive amino groups to purified goat anti-human IgG or oat anti-rabbit IgG to a flow cell of a carboxy-methyl (CM5) BIAcore™ sensor chip. Goat IgG was diluted in sodium cetate buffer, pH 4.0, and immobilized on a flow cell of a M5 chip using EDC to yield 1000 response units. Unreacted sites were blocked with ehanolamine. A flow rate of 60 μl/min was used. Multiple binding/elution cycles were performed by injection a 100 μl solution of 15 μg/mL Mu007 or Hu007 followed by human IL-1β, mouse IL-1β, rat IL-1β, cynomolgus monkey IL-1β, porcine IL-1β, human IL-1 receptor antagonist, and human IL-1α at decreasing concentrations for each cycle (e.g. 1500, 750, 375, 188, 94, 47, 23.5, 12, and 0 picomolar). Elution was performed with glycine-HCl, pH 1.5. BIAevaluation™ was used to analyze the kinetic data. Table 2 depicts the affinities of Hu007 and Mu007 for human and cynomolgus IL1β. Mouse IL-1β, rat IL-1β, IL-1 receptor antagonist, and human IL-1α did not bind to Hu007. Cynomolgus and porcein IL-1β had 100% binding to Hu007 relative to human IL-1β.

TABLE 2
Affinities of Hu007 and Mu007 for IL-1β

| | | KD |
|---|---|---|
| Antibody | Target Molecule | (Picomolar) |
| Mu007 | Human IL-1β | 6.2 |
| Hu007 | Human IL-1β | 10.2 |
| Mu007 | Cynomolgus IL-1β | 7.3 |
| Hu007 | Cynomolgus IL-1β | 10.4 |

### EXAMPLE 10

Antibody Potency

A murine cell requiring low levels of IL-1β for proliferation was used to determine the ability of Hu007 and Mu007 to neutralize human IL-1β. T1165.17 cells which are no longer in log phase growth were washed 3 times with RPMI 1640 (GibcoBRL Cat. # 22400-089) supplemented with 10% fetal calf serum (GibcoBRL Cat. # 10082-147), 1 mM sodium pyruvate, 50 μM beta mercaptoethanol, and an antibiotic/antimycotic (GibcoBRL Cat. # 15240-062). Cells were plated at 5,000 cells per well of a 96 well plate. Human IL-1β was present at a constant level of 0.3 pM and a dilution series of antibody was added. Diluted samples were added and cells were incubated for 20 hours in a 37° C./5% CO₂ incubator at which point 1 μRCi ³H-thymidine was added per well and plates incubated an additional 4 hours in the incubator. Cells were harvested and incorporated radioactivity determined by a scintillation counter. FIG. 5 illustrates inhibition of IL-1β stimulated proliferation by Mu007 and Hu007. Average IC50 values calculated from three separate experiments for Mu007 and Hu007 were 220 pM and 480 pM respectively.

### EXAMPLE 11

Neutralization of Human IL-1β in Vivo

Human IL-1β is able to bind and stimulate the mouse IL-1β receptor, leading to an elevation of mouse IL-6. Time and dose ranging experiments were undertaken to identify the optimal dose of human IL-1β and the optimal time for induction of mouse IL-6. These experiments indicated that a 3 μg/kg dose of human IL-1β and a time of 2 hours post IL-1β administration gave maximal levels of IL-6 in mouse serum. Mu007 and Hu007 were administered IV to mice one hour prior to an IP injection of human IL-1β at 27, 81 270, and 2700 μg/kg. At two hours post IL-1β administration, mice were sacrificed, and IL-6 levels were determined by ELISA. Isotype matched antibodies were used as negative controls. Mu007 and Hu007 inhibit human IL-1β induction of mouse IL-6 in a dose dependent manner beginning at 81 and 270 μg/kg with total inhibition of IL-6 indution at 2700 μg/kg.

1-39. (canceled)

40. An antibody that specifically binds mature human IL-1β wherein the antibody binds the same epitope on mature human IL-1β as mouse monoclonal antibody Mu007 or humanized antibody Hu007.

41. The antibody of claim 40 that specifically binds mature human IL-1β with an affinity constant that is within ten-fold the affinity constant of mouse monoclonal antibody Mu007 for human IL-1β.

42. The antibody of claim 40 wherein the antibody comprises at least one complementarity determining region having a sequence selected from the group consisting of SEQ ID NO:5, SEQ ID NO:6, SEQ ID NO:7, SEQ ID NO:8, SEQ ID NO:9, and SEQ ID No:10.

43. The antibody of claim 42 wherein the antibody comprises a light chain variable region having the sequence of SEQ ID NO:27.

44. The antibody of claim 42 wherein the antibody comprises a heavy chain variable region having the sequence of SEQ ID NO:28.

45. A humanized antibody which specifically binds mature human IL-1β comprising a humanized light chain which is comprised of three light chain complementarity determining regions (CDRs) having sequences that correspond to SEQ ID NO:5, SEQ ID NO:6, and SEQ ID NO:7 and a humanized heavy chain which is comprised of three heavy chain CDRs having sequences that correspond to SEQ ID NO:8, SEQ ID NO:9, and SEQ ID NO:10.

46. The humanized antibody of claim 45 comprising a light chain variable region having the sequence of SEQ ID NO:27.

47. The humanized antibody of claim 45 comprising a heavy chain variable region having the sequence of SEQ ID NO:28.

48. The humanized antibody of claim 45 comprising a light chain variable region having the sequence of SEQ ID NO:11 and a heavy chain variable region having the sequence of SEQ ID NO:15.

49. The humanized antibody of claim 48 comprising a light chain having the sequence of SEQ ID NO:13 and a heavy chain having the sequence of SEQ ID NO:17.

50. An antibody that specifically binds human IL-1β wherein the variable domains of the antibody have framework regions which correspond to one or more human immunoglobulin heavy or light chain variable domain germline sequences and complementarity determining regions (CDRs) having sequences that correspond to: SEQ ID NO:5, SEQ ID NO:6, SEQ ID NO:7, SEQ ID NO:8, SEQ ID NO:9, and SEQ ID NO:10.

51. An antibody of claim 40 comprised of complementarity determining regions (CDRs) wherein said CDRs are a modified form of the CDRs that correspond to the CDRs of antibody Mu007 wherein said modification improves binding affinity or biological activity compared to the binding affinity or biological activity of the Hu007 antibody.

52. An antibody fragment obtainable by enzymatic cleavage of the antibody of claim 40.

53. The antibody fragment of claim 52 which is a Fab or F(ab′)₂ fragment.

54. An antibody fragment obtainable by enzymatic cleavage of the antibody of claim 45.

55. The antibody fragment of claim 54 which is a Fab or F(ab′)₂ fragment.

56. An antibody fragment obtainable by enzymatic cleavage of the antibody of claim 50.

57. The antibody fragment of claim 56 which is a Fab or F(ab′)₂ fragment.

58. The antibody of claim 40, which is a single chain antibody.

59. The antibody of claim 45, which is a single chain antibody.

60. The antibody of claim 50, which is a single chain antibody.

61. The antibody of claim 40 wherein the antibody has an IgG isotype.

62. The antibody of claim 61 wherein the isotype is selected from the group consisting of IgG1 and IgG4.

63. The antibody of claim 62 wherein the isotype is IgG1.

64. The antibody of claim 45 wherein the antibody has an IgG isotype.

65. The antibody of claim 64 wherein the isotype is selected from the group consisting of IgG1 and IgG4.

66. The antibody of claim 65 wherein the isotype is IgG1.

67. The antibody of claim 50 wherein the antibody has an IgG isotype.

68. The antibody of claim 67 wherein the isotype is selected from the group consisting of IgG1 and IgG4.

69. The antibody of claim 68 wherein the isotype is IgG1.

70. The antibody of claim 40 wherein the antibody has a binding affinity for mature human IL-1β which is within 5-fold of the binding affinity of Mu007 for mature human IL-1β.

71. The antibody of claim 70, which is within 3-fold the binding affinity of Mu007.

72. The antibody of claim 45 wherein the antibody has a binding affinity for mature human IL-1β which is within 5-fold of the binding affinity of Mu007 for mature human IL-1β.

73. The antibody of claim 72, which is within 3-fold the binding affinity of Mu007.

74. The antibody of claim 50 wherein the antibody has a binding affinity for mature human IL-1β which is within 5-fold of the binding affinity of Mu007 for mature human IL-1β.

75. The antibody of claim 74, which is within 3-fold the binding affinity of Mu007.

76. The antibody of claim 40 wherein the heavy chain or light chain variable framework region has at least 80% sequence identity with the corresponding framework region of the antibody Mu007.

77. The antibody of claim 45 wherein the heavy chain or light chain variable framework region has at least 80% sequence identity with the corresponding framework region of the antibody Mu007.

78. The antibody of claim 50 wherein the heavy chain or light chain variable framework region has at least 80% sequence identity with the corresponding framework region of the antibody Mu007.

79. The antibody of claim 40 wherein the antibody has an IC50 for mature human IL-1β within 10-fold the IC50 of Mu007 for mature human IL-1β.

80. The antibody of claim 79 wherein the antibody has an IC50 within 5-fold that of Mu007.

81. The antibody of claim 80 wherein the antibody has an IC50 within 3-fold that of Mu007.

82. The antibody of claim 45 wherein the antibody has an IC50 for mature human IL-1β within 10-fold the IC50 of Mu007 for mature human IL-1β.

83. The antibody of claim 82 wherein the antibody has an IC50 within 5-fold that of Mu007.

84. The antibody of claim 83 wherein the antibody has an IC50 within 3-fold that of Mu007.

85. The antibody of claim 50 wherein the antibody has an IC50 for mature human IL-1β within 10-fold the IC50 of Mu007 for mature human IL-1β.

86. The antibody of claim 85 wherein the antibody has an IC50 within 5-fold that of Mu007.

87. The antibody of claim 86 wherein the antibody has an IC50 within 3-fold that of Mu007.

88. An isolated nucleic acid, comprising a polynucleotide encoding an antibody of claim 40.

89. The nucleic acid of claim 88 comprising one or more polynucleotides having a sequence selected from the group consisting of SEQ ID NO:1, SEQ ID NO:3, SEQ ID NO:11, SEQ ID NO:13, SEQ ID NO:15, and SEQ ID NO:17.

90. The nucleic acid of claim 89 comprising a polynucleotide having a sequence which corresponds to SEQ ID NO:13 and SEQ ID NO:17.

91. An expression vector comprising a nucleic acid according to claim 88.

92. A host cell stably transfected with the expression vector of claim 91.

93. The host cell of claim 92 wherein the host cell is selected from the group consisting of a Chinese Hamster Ovary cell, SP2/0 myeloma cell, NS0 Myeloma cell, a syrian hamster ovary cell, and an embryonic kidney cell.

94. The host cell of claim 93, which is a Chinese Hamster Ovary cell.

95. A process for producing an antibody comprising culturing the host cell of claim 94 under conditions suitable for expression of said antibody and recovering said antibody from the cell culture.

96. A pharmaceutical composition comprising the antibody of claim 40.

97. A pharmaceutical composition comprising the antibody of claim 45.

98. A pharmaceutical composition comprising the antibody of claim 50.

99. A method of treating rheumatoid arthritis or osteo-arthritis, comprising administering to a subject an effective amount of the antibody of claim 40.

100. A method of treating rheumatoid arthritis or osteo-arthritis, comprising administering to a subject an effective amount of the antibody of claim 45.

101. A method of treating rheumatoid arthritis or osteo-arthritis, comprising administering to a subject an effective amount of the antibody of claim 50.

102. A method of inhibiting the destruction of cartilage, comprising administering to a subject in need thereof an effective amount of the antibody of claim 40.

103. A method of inhibiting the destruction of cartilage, comprising administering to a subject in need thereof an effective amount of the antibody of claim 45.

104. A method of inhibiting the destruction of cartilage, comprising administering to a subject in need thereof an effective amount of the antibody of claim 50.

105. The use of the antibody of claim 40 for the manufacture of a medicament to treat a subject with rheumatoid arthritis or osteo-arthritis.

106. The use of the antibody of claim 40 for the manufacture of a medicament to inhibit cartilage destruction in a subject in need thereof.

107. The use of the antibody of claim 45 for the manufacture of a medicament to treat a subject with rheumatoid arthritis or osteo-arthritis.

108. The use of the antibody of claim 45 for the manufacture of a medicament to inhibit cartilage destruction in a subject in need thereof.

109. The use of the antibody of claim 50 for the manufacture of a medicament to treat a subject with rheumatoid arthritis or osteo-arthritis.

110. The use of the antibody of claim 50 for the manufacture of a medicament to inhibit cartilage destruction in a subject in need thereof.


SEQUENCE LISTING (28 sequences)

SEQ ID NO: 1 - DNA, 321 bp, Mus sp.
  CDS (1)..(321)
  misc_feature (24)..(34): Kabat region
gacatcaagatgacccagtctccatcttccatgtatgcatctctaggagagagagtcactatcacttgcaaggcgagtcaggacattgataggtatttaagttggttccagcagaaaccagggaaatctcctaagaccctgatctatcgtgtaaagagattggtagatggggtcccatcaaggttcagtggcagcgcatctgggcaagattattctctcaccatcagcagcctgcagtatgaagatatgggaatttattattgtctacagtatgatgagtttccgtacacgttcggaggggggaccaagctggaaataaaa

SEQ ID NO: 2 - protein, 107 aa, Mus sp.
DIKMTQSPSSMYASLGERVTITCKASQDIDRYLSWFQQKPGKSPKTLIYRVKRLVDGVPSRFSGSASGQDYSLTISSLQYEDMGIYYCLQYDEFPYTFGGGTKLEIK

SEQ ID NO: 3 - DNA, 360 bp, Mus sp.
  CDS (1)..(360)
  misc_feature (31)..(35): Kabat region
caggttcagctggtgcagtctggagctgaggtgaagaagcctggggcctcagtgaaggtgtcctgcaaggtgtctggctacacattcagtaggtattggatagagtgggttagacaggcacctggaaaaggccttgagtggattggagagattttacctggaaatggaaatattaactacaatgagaagttcaagggcaaggccacaatcacagcagatacatccacagatacagcctacatggaactcagcagcctgaggtctgaggacacagccgtctattattgttcaacaatctactatgattacgaccaggggtttacttactggggccaagggactctggtcactgtttctgca

SEQ ID NO: 4 - protein, 120 aa, Mus sp.
QVQLVQSGAEVKKPGASVKVSCKVSGYTFSRYWIEWVRQAPGKGLEWIGEILPGNGNINYNEKFKGKATITADTSTDTAYMELSSLRSEDTAVYYCSTIYYDYDQGFTYWGQGTLVTVSA

SEQ ID NO: 5 - protein, 11 aa, Artificial Sequence
  Humanized antibody light chain CDR1
KASQDIDRYLS

SEQ ID NO: 6 - protein, 7 aa, Artificial Sequence
  Humanized antibody light chain CDR2
RVKRLVD

SEQ ID NO: 7 - protein, 8 aa, Artificial Sequence
  Humanized antibody light chain CDR3
LQYDEFYT

SEQ ID NO: 8 - protein, 5 aa, Artificial Sequence
  Humanized antibody heavy chain CDR1
RYWIE

SEQ ID NO: 9 - protein, 17 aa, Artificial Sequence
  Humanized antibody heavy chain CDR2
EILPGNGNINYNEKFKG

SEQ ID NO: 10 - protein, 11 aa, Artificial Sequence
  Humanized antibody heavy chain CDR3
IYYDYDQGFTY

SEQ ID NO: 11 - DNA, 321 bp, Artificial Sequence
  Humanized antibody light chain variable region
gacatccagatgacccagtctccatcttccctgtctgcatctgtaggagacagagtcactatcacttgcaaggcgagtcaggacattgataggtatttaagttggttccagcagaaaccagggaaagctcctaagtccctgatctatcgtgtaaagagattggtagatggggtcccatcaaggttcagtggcagcgcatctgggacagattatactctcaccatcagcagcctgcagcctgaagatttcgcaacctattattgtctacagtatgatgagtttccgtacacgttcggacaggggaccaagctggaaataaaa

SEQ ID NO: 12 - protein, 107 aa, Artificial Sequence
  Synthetic Construct
DIQMTQSPSSLSASVGDRVTITCKASQDIDRYLSWFQQKPGKAPKSLIYRVKRLVDGVPSRFSGSASGTDYTLTISSLQPEDFATYYCLQYDEFPYTFGQGTKLEIK

SEQ ID NO: 13 - DNA, 642 bp, Artificial Sequence
  Humanized antibody light chain variable region
gacatccagatgacccagtctccatcttccctgtctgcatctgtaggagacagagtcactatcacttgcaaggcgagtcaggacattgataggtatttaagttggttccagcagaaaccagggaaagctcctaagtccctgatctatcgtgtaaagagattggtagatggggtcccatcaaggttcagtggcagcgcatctgggacagattatactctcaccatcagcagcctgcagcctgaagatttcgcaacctattattgtctacagtatgatgagtttccgtacacgttcggacaggggaccaagctggaaataaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 14 - protein, 214 aa, Artificial Sequence
  Synthetic Construct
DIQMTQSPSSLSASVGDRVTITCKASQDIDRYLSWFQQKPGKAPKSLIYRVKRLVDGVPSRFSGSASGTDYTLTISSLQPEDFATYYCLQYDEFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 360 bp, Artificial Sequence
  Humanized antibody heavy chain variable region
caggttcagctggtgcagtctggagctgaggtgaagaagcctggggcctcagtgaaggtgtcctgcaaggtgtctggctacacattcagtaggtattggatagagtgggttagacaggcacctggaaaaggccttgagtggattggagagattttacctggaaatggaaatattaactacaatgagaagttcaagggcaaggccacaatcacagcagatacatccacagatacagcctacatggaactcagcagcctgaggtctgaggacacagccgtctattattgttcaacaatctactatgattacgaccaggggtttacttactggggccaagggactctggtcactgtttcttct

SEQ ID NO: 16 - protein, 120 aa, Artificial Sequence
  Synthetic Construct
QVQLVQSGAEVKKPGASVKVSCKVSGYTFSRYWIEWVRQAPGKGLEWIGEILPGNGNINYNEKFKGKATITADTSTDTAYMELSSLRSEDTAVYYCSTIYYDYDQGFTYWGQGTLVTVSS

SEQ ID NO: 17 - DNA, 1350 bp, Artificial Sequence
  Humanized antibody full-length heavy chain region
caggttcagctggtgcagtctggagctgaggtgaagaagcctggggcctcagtgaaggtgtcctgcaaggtgtctggctacacattcagtaggtattggatagagtgggttagacaggcacctggaaaaggccttgagtggattggagagattttacctggaaatggaaatattaactacaatgagaagttcaagggcaaggccacaatcacagcagatacatccacagatacagcctacatggaactcagcagcctgaggtctgaggacacagccgtctattattgttcaacaatctactatgattacgaccaggggtttacttactggggccaagggactctggtcactgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 18 - protein, 450 aa, Artificial Sequence
  Synthetic Construct
QVQLVQSGAEVKKPGASVKVSCKVSGYTFSRYWIEWVRQAPGKGLEWIGEILPGNGNINYNEKFKGKATITADTSTDTAYMELSSLRSEDTAVYYCSTIYYDYDQGFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 66 bp, Artificial Sequence
  Humanized antibody light chain signal peptide
atggacatgaggacccctgctcagtttcttggaatctttttcttctggtttccaggtatcagatgt

SEQ ID NO: 20 - protein, 22 aa, Artificial Sequence
  Synthetic Construct
MDMRTPAQFLGIFFFWFPGIRC

SEQ ID NO: 21 - DNA, 57 bp, Artificial Sequence
  Humanized antibody heavy chain signal peptide to precede SEQ ID NO 3
atggaatggacctgggtctttctcttcctcctgtcagtaactgcaggtgtccactcc

SEQ ID NO: 22 - protein, 19 aa, Artificial Sequence
  Synthetic Construct
MEWTWVFLFLLSVTAGVHS

SEQ ID NO: 23 - DNA, 66 bp, Artificial Sequence; identical to SEQ ID NO: 19
  Humanized antibody full-length chain signal peptide to precede SEQ ID NO 11, 13 or 27

SEQ ID NO: 24 - protein, 22 aa, Artificial Sequence
  Synthetic Construct
MDMRTPAQFLGIFFFWFPGIRC

SEQ ID NO: 25 - DNA, 57 bp, Artificial Sequence; identical to SEQ ID NO: 21
  Humanized antibody full-length heavy chain signal peptide to precede SEQ ID NO 15, 17 or 28

SEQ ID NO: 26 - protein, 19 aa, Artificial Sequence
  Synthetic Construct
MEWTWVFLFLLSVTAGVHS

SEQ ID NO: 27 - protein, 107 aa, Artificial Sequence
  light chain variable region, Formula I
AIXMTQXPSSXXASXGXRVTITCKASQDIDRYLSWFQQKPGKAPKXLIYRVKRLVDGVPSRFSGSXSGXDYTLTISSLQPEDFATYYCLQYDEFPYTFGQGTKLEIK

SEQ ID NO: 28 - protein, 120 aa, Artificial Sequence
  heavy chain variable region, Formula II
XVQLVQSGAEVKKPGASVKVSCKXSGYTFXRYWIEWXRQAPGXGLEWXGEILPGNGNINYNEKFKGXXTXTADXSXXTAYMELSSLXSEDTAVYYCSTIYYDYDQGFTYWGQGTLVTVSS